(19) United States
(12) Patent Application Publication
(10) Pub. No.: US 2026/0047935 A1
(43) Pub. Date: Feb. 19, 2026

(54) CASE-LIKE DEVICE AND PROSTHETIC COMPONENT EQUIPPED WITH SUCH DEVICE

(71) Applicant: TECRES S.p.A., Sommacampagna (Verona) (IT)

(72) Inventors: Renzo SOFFIATTI, Nogara (VR) (IT); Giovanni FACCIOLI, Monzambano (MN) (IT)

(21) Appl. No.: 19/368,544

(22) Filed: Oct. 24, 2025

Related U.S. Application Data
(62) Division of application No. 17/616,856.

(30) Foreign Application Priority Data
Jul. 17, 2019 (IT) 102019000012201

(51) Int. Cl. A61F 2/30; A61F 2/36; A61F 2/38; A61F 2/40

(52) U.S. Cl. CPC A61F 2/30734; A61F 2/36; A61F 2/3804; A61F 2/389; A61F 2/40; A61F 2002/30113; A61F 2002/30131; A61F 2002/30156; A61F 2002/3021; A61F 2002/30224; A61F 2002/30276; A61F 2002/30332; A61F 2002/30354; A61F 2002/30359; A61F 2002/30738; A61F 2002/30784; A61F 2002/30884; A61F 2310/00011; A61F 2310/00353

(57) ABSTRACT

Prosthetic component provided with a stem, adapted to be implanted in use at a bone of a patient, such as for example a tibial and/or femoral component of a knee prosthesis or a femoral component of a hip prosthesis or a humeral component of a shoulder prosthesis, or a component of an elbow prosthesis, including a device adapted to externally cover and/or to house at its interior at least the stem of the prosthetic component, wherein the device is in use placed between the prosthetic component and the bone of the patient, suitable to stably constrain in use the prosthetic component to the bone of the patient and to prevent the direct contact between prosthetic component and bone of the patient.

### TECHNICAL FIELD OF THE INVENTION

The present invention refers to a case-like device to be inserted under and/or around a prosthetic component, such as a tibial or femoral or humeral or elbow component of a permanent prosthesis, and in particular under the stem of the latter, in such a way that such case-like device, once assembled with the prosthetic component and/or the stem thereof, is suitable to be implanted at a bone of the patient such as the tibial or femoral bone of a knee joint, the femoral bone of a hip joint or the humeral bone of a shoulder joint, the elbow bone, etc.

Furthermore, the present invention refers also to a system or kit comprising a prosthetic component, such as for example a tibial and/or femoral component of a knee prosthesis, or a femoral component of a hip prosthesis or the humeral component of a shoulder prosthesis, or a component of an elbow prosthesis, equipped with a such case-like device.

### STATE OF THE ART

As it is known, articular prostheses, after being implanted in the human body, despite being so-called “permanent” devices, may require their removal, in case, for example, of an infection arisen in the implantation site.

Another cause that may determine the removal of a prosthesis or of one of its components is that the fixed constraint of the connection between the same and the bones of the human body at which it has been implanted is lost.

Such an eventuality occurs, for example, with reference to the cemented knee prostheses, because the tibial component and/or the femoral component can “unstick” from the bone cement that is used for its connection to tibial bone.

The same applies to the femoral component of a hip prosthesis or for the humeral component of a shoulder prosthesis.

Furthermore, the direct contact between the metal with which the prosthesis is made and the patient's bone (for example in areas where the bone cement used to cement the prosthesis at the implant site has not arrived) can cause a phenomenon known as bone reabsorption (Stress Shielding). This phenomenon causes a real dissolution of the bone tissue with reduction of its cortical and trabecular mass, and the consequence is that the bone reduces its strength and the ability to withstand the loads transmitted thereto while the implanted prosthesis loses stability, detaches from the implant site and must be removed.

It is known indeed that a lasting result of a cemented implant depends on several factors, among which of main importance is the application way of the cement. The optimal cementation of a stem of a prosthesis foreseen to coat the same uniformly with a regular layer of approximately 2 mm of bone cement, so as to evenly distribute—by the layer of cement—the forces coming from the metal stem of the prosthesis.

If, however, the coating of cement has some gaps or windows, the metal stem can touch the bone with consequent—in few months—resorption of the bone portion in contact with the metal of the stem (Stress Shielding) and mobilization—and therefore failure—of the implant.

In conclusion, therefore, in order that a cemented prosthetic stem is perfectly affixed, it is necessary it possesses a regular, uniform, continuous cement mantle and having the correct thickness. However, these conditions are not easily obtainable by current cementation techniques. The positioning of the stem inside a diaphysis filled with cement takes place in fact “blindly” and the tip of the stem very often goes in touch with the bone, determining the serious consequences described above.

In the past, some attempts have been developed to equip the prosthetic stems with centering rings placed near the tip, but it is anyway a solution which is not entirely able to overcome the above-mentioned drawbacks.

All these disadvantages determine that the contact surfaces between prosthesis and bone cement, or as well as between bone and bone cement, are the weakest points of the implant and which more often cause the need to replace the permanent prosthesis.

The patent application EP1872747 discloses a hinged knee prosthesis.

The U.S. Pat. No. 7,070,622 discloses an articular prosthesis for knee, humerus or hip.

The patent application EP2042132 discloses a tibial prosthesis.

There is therefore the need for a solution that completely avoids any possible contact between the metal stem and the bone of the patient.

### SUMMARY OF THE INVENTION

A purpose of the present invention is to improve the state of the art.

Another purpose of the present invention is to provide a case-like device suitable to be positioned externally to a prosthetic component, and/or to the stem of a prosthetic component such as the tibial or femoral or humeral component of a prosthesis, and to ensure a layer of a biocompatible material (constituted by the device itself) to which the bone cement correctly adheres in a stable and long-lasting way.

A further purpose of the present invention is to provide a case-like device which ensures a cover layer of the metal stem of a prosthesis (different from the metal material with which the prosthetic stem is made) having an optimal minimum thickness on the whole surface of the stem itself.

A still further purpose of the present invention is to provide a kit composed of a prosthetic component such as a tibial and/or femoral component of a knee prosthesis, or a femoral component of a hip prosthesis or even the humeral component of a shoulder prosthesis, and a case-like device suitable to ensure a layer of a biocompatible material (different from the metal material with which the prosthesis is made) to which the bone cement correctly adheres in a stable way, with consequent stable and long-lasting constraint to the bone of the patient.

A further purpose of the present invention is to provide a kit wherein the prosthetic component equipped with a case-like device according to the present invention is suitable to completely avoid the risk of accidental contact between the prosthetic stem and the patient's bone.

According to one aspect of the present invention, a kit is provided equipped with a prosthetic component, possibly a tibial and/or femoral component of a knee prosthesis, or a femoral component of a hip prosthesis hi or a humeral component of a shoulder prosthesis, and with a case-like device according to the present application.

According to another aspect of the present invention, a case-like device is provided according to the present application.

The present application refers to preferred and advantageous embodiments of the invention.

### BRIEF DESCRIPTION OF THE DRAWINGS

Further characteristics and advantages of the present invention will be more evident from the detailed description of a preferred but not exclusive embodiment of a device for a prosthetic component such as a tibial component of a knee prosthesis, illustrated as a non-limiting example in the enclosed drawing tables in which:

FIG. 1 is a perspective view of a tibial component of a traditional type knee prosthesis;

FIG. 2 is a perspective view of a case-like device according to a version of the present invention;

FIG. 3 is perspective view slightly from below of a prosthetic component of a knee prosthesis assembled with the case-line device of FIG. 2;

FIG. 4 is a lateral view of the case-like device of FIG. 2;

FIG. 5 is a view from below of the case-like device of FIG. 2;

FIG. 6 is a lateral view of the case-like device of FIG. 2;

FIG. 7 is a sectional front view of the case-like device of FIG. 2, taken along the section plane A-A-of FIG. 6;

FIG. 8 is a perspective view of a femoral component of a hip prosthesis and of a case-like device according to a version of the present invention;

FIG. 9 is a perspective view slightly from above of a case-like device according of FIG. 8;

FIG. 10 is a rear view of a femoral component of a hip prosthesis and of the case-like device of FIG. 8, in an assembled version;

FIG. 11 is a lateral view of the prosthetic component of FIG. 8; and

FIG. 12 is a lateral view of the assembled version of the femoral component of a hip prosthesis and of a case-like device of FIG. 10.

### DETAILED DESCRIPTION OF THE INVENTION

With reference to the enclosed figures, with 1 a component of a prosthesis is overall indicated.

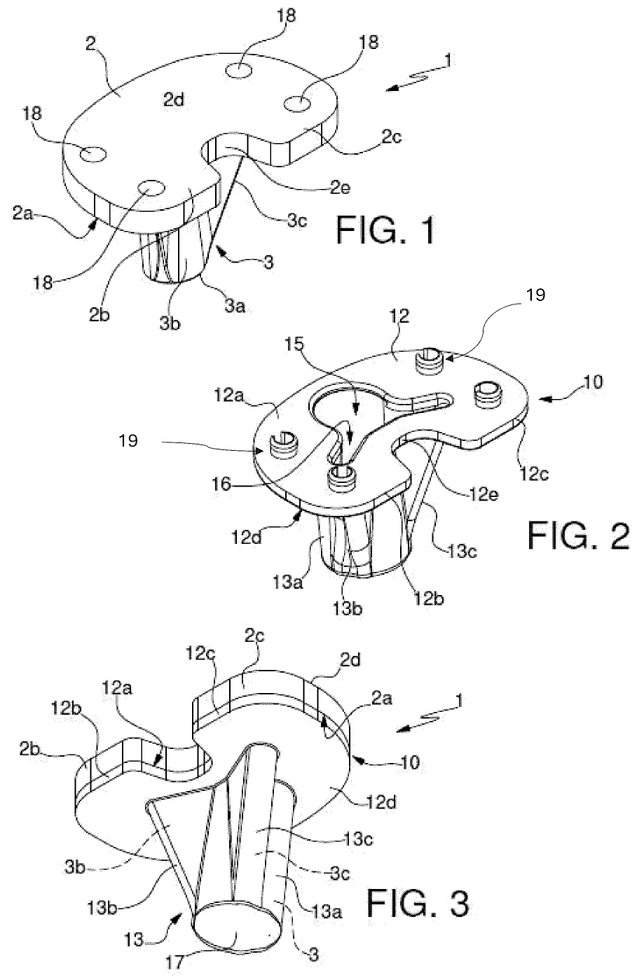

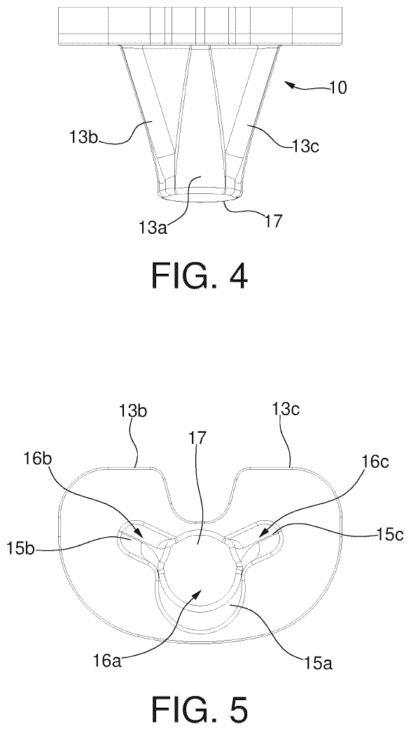

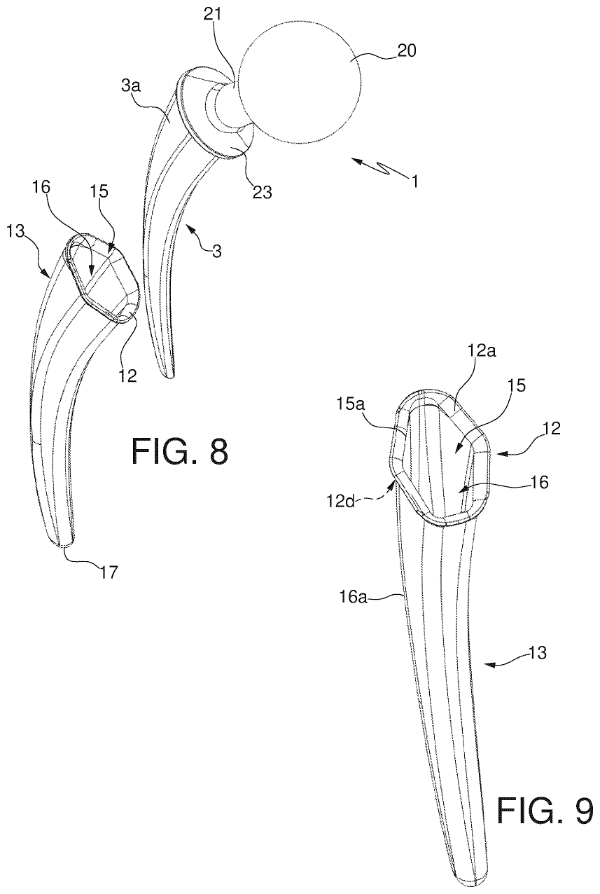

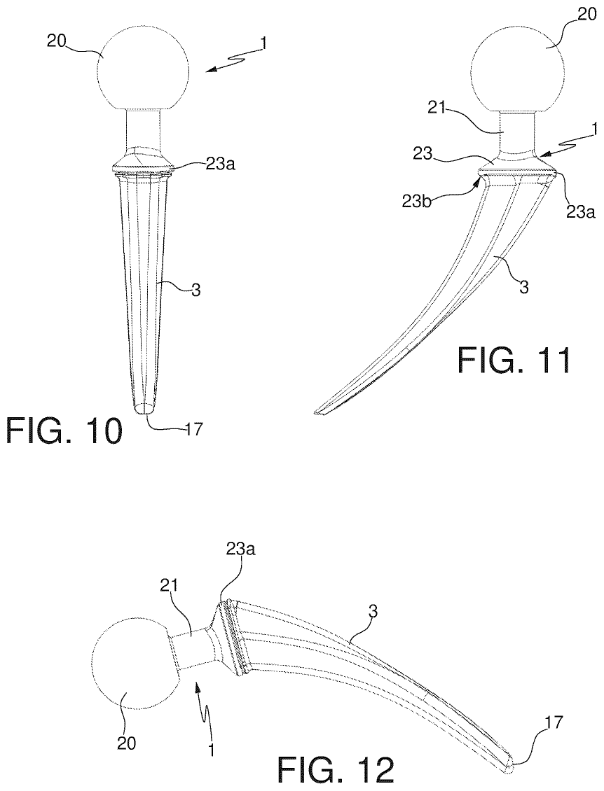

For example, the component 1 can be a tibial component of a prosthesis (for example total) of the knee, as shown in FIGS. 1 to 7, and/or a femoral component of a knee prosthesis, and/or a femoral component of a hip prosthesis (as shown in FIGS. 8 to 12) or yet a humeral component of a shoulder prosthesis (version not shown) or a component of an elbow prosthesis.

The prosthetic component 1, therefore, is a component of a prosthesis of an anatomical district of the human body equipped with a stem which in use is cemented to the bone of the patient.

Therefore, although in the description which follows and in the enclosed drawings respectively a tibial component of a knee prosthesis and a femoral component of a hip prosthesis will be described and shown, the present invention is to be considered valid also for other prosthetic components, to be implanted also in other districts of the human body with respect to the indicated joints.

With “proximal” in the present description it is meant a component or element closer to an end of a bone of a patient or closer to another component or element while the term “distal” it is meant a more inner component in a bone of a patient and/or farther away from one end of a bone of a patient or farther away from another component or element. The prosthetic component 1 is made by a stem 3.

Considering the case in which the prosthetic component 1 is a tibial component of a knee joint, it also includes a tibial plate 2, in addition to the stem 3.

The tibial plate 2 extends in a pattern substantially parallel to a transverse plane of the body human passing at the proximal end or proximal epiphysis of the tibia.

The tibial plate has a substantially C-shaped conformation, in which the two free ends 2b, 2c of the C are placed at the back of the knee.

The stem 3, in this version of the invention, has a portion 3a (or first portion 3a) substantially cylindrical which extends from a lower face 2a of the tibial plate 2 in a substantially central position. The stem 3 and/or its portion 3a extend at the medullary canal of the patient's tibia.

The stem 3, in further versions, can have a substantially conical or frustoconical or pyramid frustum shape.

From the portion 3a at least two lateral wings 3b, 3c depart, having a substantially triangular shape, for example with a right angle. In particular, these lateral wings 3b, 3c each has one long side that adheres and/or is constrained substantially to the vertical extension in use of the portion 3a, a short side that adheres and/or is constrained substantially to the lower face 2a of the tibial plate 2 and at the end the diagonal which extends outwardly with respect to the portion 3a of the stem 3.

In particular, the short sides of the lateral wings 3b, 3c protrude toward the free ends 2b, 2c of the tibial plate 2. The angle enclosed between the lateral wings 3b, 3c (considering that subtended at the back portion of the knee) is less than 180°or better between 45°and 135°. However, this angle may vary according to the patient's needs.

The lateral wings, therefore, have respectively three vertices. Two vertices (those placed at the ends of the long side of the triangle) are connected and/or constrained to the portion 3a, one of these two vertices (the most proximal) also corresponds to one of the ends of the short side of the triangle. Therefore, the two vertices of each lateral wing which are placed at the ends of the short side of the triangle are connected and/or constrained to the lower face 2a of the tibial plate 2.

Therefore, one of those vertices is connected and/or constrained both to the portion 3a and to the lower face 2a of the tibial plate 2, being placed at the contact point between such two elements.

The tibial plate 2 also comprises an upper face 2d which faces toward the femoral component (not shown) of the knee prosthesis.

The present invention includes a case-like or shell-like device 10. The device 10 is suitable to externally cover at least the stem 3 of the prosthesis component 1. In the version illustrated in the FIGS. 1 to 7, the device 10 covers also face the lower face 2a of the tibial plate 2 of the prosthetic component 1, which in such case is the tibial component of a knee prosthesis.

For this reason, in this version, the device 10 may be defined under-tibial device.

The device 10 is therefore able to house at least the stem 3 of the prosthetic component 1 inside it.

The device 10 therefore, in use, is an intermediate device that is positioned between the prosthetic component 1 and the patient's bone so that the latter is not in direct contact with the prosthetic component 1 at any point. In this way, since the prosthetic component 1 and/or the stem 3 is made of a metallic material while the device 10 is made of a biocompatible material other than metal, the risk of bone resorption is avoided and, at the same time, the prosthetic component 1 and/or stem 3 is constrained to the bone at the implant site.

The device 10 of the present invention therefore acts as a casing or shell that externally covers at least part of the prosthetic component 1 and/or at least the stem 3 of the prosthetic component 1.

Furthermore, the device 10 ensures the presence of a constant thickness along the entire surface of at least the stem 3 of the prosthetic component 1, so as to ensure a constant distance between the stem 3 and the patient's bone.

The prosthetic component 1, in fact, is made of a metal material while the device 10 is made of a biocompatible material different from the metal material of the prosthetic component 1.

In fact, therefore, the device 10 in use will be in contact with the bone interface of the implantation site, completely preventing that the contact with the latter occurs by the prosthetic component 1.

As regards the prosthetic component 1, therefore, the bone interface consists exclusively of the device 10, since the latter is interposed between the prosthetic component and the patient's bone.

Naturally, there will be some adhesion between the prosthetic component 1 and the device 10, also implemented by the bone cement in the liquid or fluid state used for cementing the prosthetic component itself, in order to determine a certain stability to the implant itself and avoid any play between the device 10 and the prosthetic component 1 (or better the stem 3).

The device 10 is made of a biocompatible plastic material, such as bone cement and/or PMMA, i.e. a material (solid) similar to bone cement in the liquid and/or viscous state that is traditionally used to bind the prostheses to the patient's bone. This affinity therefore allows perfect adhesion between the material of which the device 10 is made and the liquid and/or viscous bone cement used for bonding the prosthesis to the bone. The device 10 is however made of a material other than metal, in order to overcome the drawbacks mentioned above.

Furthermore, thanks to the fact that the device 10 already constitutes a layer of biocompatible material of the type indicated above, it requires a smaller quantity of cement needed to cement the stem 3 (it is in fact already pre-coated by the minimum optimal layer determined from the case-like device 10). Furthermore, this smaller amount of cement for implant fixation causes less thermal damage to the patient's bone, which usually to a greater or lesser extent is affected by contact with the bone cement in the liquid and/or fluid state and/or by temperatures which arise from its polymerization and hardening reaction.

Therefore, the device 10 has a surface wall having a thickness of at least 2 mm or between 0.5 mm and 5 mm.

Considering the version illustrated in FIGS. 1 to 7, the device 10 comprises—in a way substantially corresponding to the conformation of the prosthetic component 1, when he latter is the tibial component of a knee prosthesis—a tibial base 12 having a substantially C-shaped form and equipped with an upper surface 12a and a lower surface 12d, as well as with two free ends of the C, 12b and 12c, facing the rear of the knee.

The tibial base 12 is adapted in use to come into contact and/or to be constrained with the lower face 2a of the tibial plate 2.

Therefore, the conformation and dimensions of the tibial base 12 substantially correspond to those of the tibial plate 2 (as visible in the assembled version of FIG. 3). The tibial base 12 has a recess 12e which determines its C-shaped conformation thereof and which separates its free ends 12b and 12c. This recess corresponds to a recess 2e of the tibial plate 2 which performs the same function.

The Device 10 Also Comprises a Shaft 13.

In the version relating to the tibial component of a knee prosthesis, the shaft 13 is provided with a part 13a (or first part 13a) having a substantially tubular conformation. In particular, the part 13a has a side wall having a substantially tubular conformation inside which a first space 16a, for example cylindrical, is enclosed. Part 13a has a section (taken along a plane parallel to the transverse plane of the human body) which is substantially circular or oval, or square with rounded edges, or polygonal with rounded edges, etc.

The shaft 13 departs from the lower surface 12d of the tibial base 12, in a direction substantially perpendicular to the same, in order to enter the medullary canal of the patient's tibial bone.

Part 13a also includes at least two lateral protrusions 13b, 13c, having—in one version of the invention—a substantially triangular shape, for example at right angle. The lateral protrusions 13b, 13c have a conformation and an arrangement substantially corresponding to that of the lateral wings 3b, 3c, but of slightly larger dimensions, so as to be able to house the wings 3b, 3c inside them (and/or inside a respective space 16b, 16c having a conformation corresponding to that of the wings 3b, 3c).

The same applies to the part 13a of the shaft 13, which has slightly larger dimensions than those of the portion 3a of the stem 3, in order to be able to house the latter inside it. For this purpose, the device 10 according to the present invention comprises an opening 15. The opening 15 is an access opening that allows the insertion of the stem 3 of the prosthetic component 1 in the shaft 13 of the case-like or shell-like device 10.

In particular, as visible in FIGS. 7 and 9, the opening 15 subtends a cavity 16 present inside the device 10.

In the version in which the prosthetic component 1 is a tibial component of a knee prosthesis, the opening 15 affects at least the tibial base 12 and the cavity 16 affects at least the tibial base 12 and the part 13a of the shaft 13 of the device 10.

The opening 15, in general, comprises a zone 15a (or first zone 15a), having a substantially circular shape and dimensions slightly greater than those of the cross section of the first portion 3a of the stem 3, which in use is inserted inside the device 10 through the zone 15a.

Considering the tibial version of the prosthetic component 1, the opening 15 also has at least two lateral openings 15b, 15c, visible for example in FIG. 5, which subtend respective hollow lateral portions suitable for inserting and housing in use the lateral wings 3b, 3b of the stem 3.

In this way, the structure is stiffened and is able to better withstand the loads to which it is subjected, and it is avoided that the prosthetic component 1 can rotate with respect to the device 10.

The lateral openings 15b, 15c have a lobed and/or elongated conformation, having a length equal to or slightly greater than the maximum transverse dimension of the lateral wings 13b, 13c and a width equal to or slightly greater than the thickness of the lateral wings 13b, 13c.

The cavity 16, therefore, at the zone 15a, has a space 16a, (or first space 16a) having a substantially cylindrical shape and slightly larger dimensions than those of the portion 3a which in use is housed inside the space 16a of the cavity 16.

In its inner part, the first space 16a has a substantially circular cross section, possibly corresponding to that of the zone 15a and/or of the portion 3a.

The cavity 16 also comprises, at the openings 15b, 15c, at least two lateral hollow portions 16b, 16c, which extend inside the lateral protrusions 13b, 13c of the shaft 13 of the device 10 according to the present invention, in the version in which the prosthetic component 1 is a tibial component of a knee prosthesis.

The hollow lateral portions 16b, 16c follow the course of the lateral protrusions 13b, 13c and of the lateral wings 3b, 3c and therefore become thinner, i.e. their width decreases, moving towards the distal end of the same.

Therefore, the lateral wings 3b, 3c, the lateral openings 15b, 15c, the at least two hollow lateral portions 16b, 16c (and possibly the lateral protrusions 13b, 13c) substantially have the same positions and configurations, but slightly different sizes, given that the lateral wings 3b, 3c in use are inserted inside the lateral openings 15b, 15c and are housed inside the at least two lateral hollow portions 16b, 16c, which in turn are positioned inside the lateral protrusions 13b, 13c.

As can be seen, for example, in FIG. 5, the opening 15 presents in plan view from above a substantially trilobed conformation, in which the lobes are formed by the first zone 15a and by the lateral openings 15b, 15c.

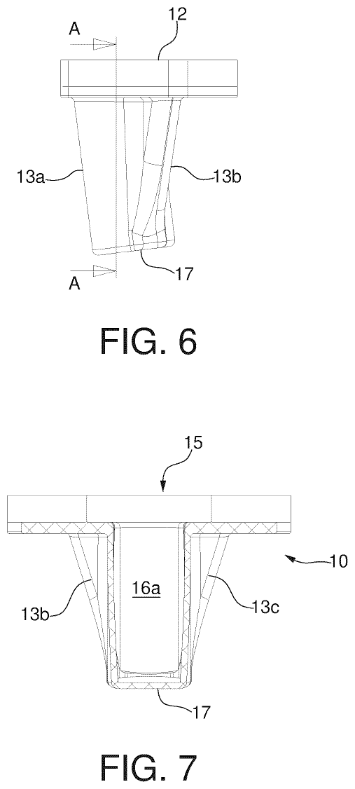

As can be seen in FIGS. 5 and 6, the shaft 13 of the device 10 has a slightly inclined extension towards the rear of the knee. Furthermore, it can be seen the closing base 17 of the part 13a is rearwardly inclined upwards with respect to the tibial base 12 of the device itself.

The closing base 17 is placed in use in the innermost area of the medullary canal and constitutes the distal end of the shaft 13.

The closing base 17 can preferably be made of the same material that constitutes the device 10 and acts as a closure both for the part 13a of the shaft 13 and for the space 16a of the cavity 16.

This inclination naturally corresponds to that the stem 3 of the prosthetic component 1 may have, so as to allow its easy insertion inside the shaft 13 of the device 10.

The closing base 17, in at least one version of the present invention, is substantially perpendicular to the side wall of the part 13a of the shaft 13 while the side wall is inclined with respect to the tibial base 12 or better to its lower surface 12d.

In one version of the invention, the lateral protrusions 13b, 13c have a vertical extension (in use) slightly lower than that of the part 13a, and their terminal or distal zone, located near the base 17, is closed, not thus allowing the lateral hollow portions 16b, 16c to be opened at their distal end.

In one version of the invention, the lower surface 12d of the tibial base 12 can have roughness and/or undercuts and/or hollow cells, in order to better adhere and more effectively retain the bone cement which is used to constrain the whole to the patient's tibial bone.

Such roughness and/or undercuts and/or cells could also be present at the external wall of the shaft 13 of the device 10, with the same purposes.

In the version illustrated in FIGS. 8 to 12, in which the prosthetic component 1 is a femoral component of a hip prosthesis, it is equipped with a stem 3 and a spherical or hemispherical or spherical cap head 20.

The stem 3 is designed to be inserted in use in the medullary canal of the bone, for example of the hip joint, while the head 20 is designed in use to articulate in the joint, for example in the acetabulum of the hip joint.

The stem 3 and the head 20 are connected by a neck structure 21, having for example a substantially cylindrical or truncated cone or pyramid shape.

The neck-like structure 21 has a first end 21a connected and/or constrained (in a fixed and/or adjustable way) to the head 20 and/or to a base present in the head 20.

The neck-like structure 21 also has a second end 21b, opposite to the first and connected and/or constrained, preferably in a fixed manner forming a single body, to the stem 3. The stem 3 has one end of connection 23, suitable in use to be connected and/or constrained to the neck 21, and a distal end 22, suitable in use to be inserted deep into the medullary canal of the bone.

As visible for example from FIG. 11, the connection end 23 departs from the neck 21 outwards in all directions and therefore has a truncated cone or pyramid shape.

The connection end 23 then reaches with its maximum extension at the stem 3 itself, and/or at a portion 3a thereof, which is substantially tapered towards the distal end 22 and/or has a substantially conical or truncated cone or pyramid conformation.

At the junction between the connection end 23 and the stem 3 (and/or portion 3a), there is a perimetric flange 23 a that extends outwards of the femoral component itself.

The perimeter flange 23 a therefore has a greater lateral extension than that of the stem 3 and is therefore projecting externally with respect to the latter. There perimetric flange 23a therefore determines a lower face 23b illustrated for example in FIG. 11.

In this version of the invention, the device 10 has a shaft 13 equipped with a part 13a having a substantially tapered conformation towards the closing base 17 and/or having a substantially conical or truncated cone or pyramid conformation, i.e. substantially corresponding to the stem 3 but slightly larger in size, so as to be able to house the stem 3 itself and/or its portion 3a inside it.

At the opening 15 (and/or at the zone 15a of the opening 15), the device 10 has a flanged base 12 that extends outwards with respect to the opening 15.

This base 12 has an upper surface 12a and a lower surface 12d, opposite the upper surface 12a.

The base 12 has a lateral extension substantially corresponding to or slightly greater than the perimetric flange 23a of the stem 3.

The upper surface 12a is adapted in use to abut and/or to contact the perimetric flange 23a, and/or with its lower face 23b.

The lower surface 12d is suitable in use to abut and/or to come into contact and/or to be constrained to the patient's bone, thus completely avoiding contact between bone and material of the prosthetic component 1.

The conformation of the cavity 16 and/or of its space 16a corresponds to that of the shaft 13.

As can be seen from FIGS. 11 and 12, the stem 3 of the prosthetic component 1 is laterally inclined and/or curved. Similarly, the device 10 has the same inclination and/or curvature.

The closing base 17 of the part 13a can therefore be inclined or not with respect to the wall of the part 13a of the shaft 13.

In one version of the invention, the device may have an internal metal core (not shown in the figures), which is completely immersed in the biocompatible material that constitutes the device 10 itself.

In an alternative version, the device 10 can be completely made of the materials indicated above, without providing an internal metal core.

Furthermore, the device 10 may or may not be impregnated with at least one active substance, such as an antibiotic or another medical agent, in order to treat an ongoing infection or other pathology in the surgical site.

According to a still further version, the device 10 can be porous and/or equipped with through holes, able to make the bone cement that is used to constrain the prosthetic component 1 to the patient's bone pass through them. In this case, the support function is not entrusted to the device 10, which therefore can have a high degree of holes and/or porosity. This function, in fact, is supported by the prosthetic component 1 itself.

In use, in fact, a very fluid bone cement can be used inside the device 10 and/or the cavity 16 (possibly inserted through the opening 15), which protrudes from the pores and/or through holes, constraining the device 10 to the patient's bone. In conjunction with this, the bone cement present inside the device 10 binds the latter to the prosthetic component 1.

Advantageously, the device 10 is preformed and/or formed of a rigid material, with predetermined sizes and thicknesses based on the type of prosthetic component 1 that must be housed.

In order to ensure an optimal connection of the device 10 to the prosthetic component 1, the latter may have housing seats 18 for corresponding connection means 19 which are present in the device 10, or vice versa.

In particular, as can be seen in FIGS. 1 and 2, the housing seats 18 (four in the illustrated version, arranged at the outermost corners of the tibial plate 2 and/or two positioned at the front portion in use of the tibial plate 2 and other two substantially positioned at the free ends 2b, 2c, even if other positions or numbers can also be provided, especially for different conformations of the prosthetic component 1) are shaped as through openings arranged along an axis parallel to the longitudinal axis of the human body and/or in the vertical direction in use, in the thickness of the tibial plate 2. Alternatively, these housing seats 18 can be shaped as openings that are not through but open at the lower face 2a of the tibial plate 2.

The connection means 19, on the other hand, are housed inside the housing seats 18 (in this sense they correspond to and/or complement the latter) and can have a substantially pin, cylinder, semi-cylinder, clip, C, ring or tubular shape, etc. In particular, the connection means 19 are inserted inside the housing seats 18 and determine with the latter a constraint and/or a reference that allows adequate adhesion and/or constraint between the two involved components.

The cross section of the housing seats 18 is substantially corresponding and slightly larger than the cross section of the connection means 19, so that the latter can be housed (possibly in a removable way) inside the housing seats 18.

In the illustrated version, the housing seats 18 have a substantially circular cross section while the connection means 19 have a substantially C-shaped cross section.

In the illustrated version, the connection means 19 are positioned at the base 12 and/or at its upper surface 12a and/or protrude from the latter towards the prosthetic component.

As said, it would be possible to have the connection means 19 protruding from the lower face 2a of the tibial plate 2 and housing seats 18 whose opening is positioned at the upper surface 12a of the tibial base 12 and extending inside the latter.

In conclusion, the present invention refers, as indicated above, to a kit or system, or in any case to an assembly formed by prosthetic component 1 and case-like or shell-like device 10. In fact, in use, they are implanted together, in order to avoid-as mentioned-the contact of the prosthetic component material with the patient's bone, thanks to the interposition of the device 10.

The kit may also possibly include bone cement in a fluid or liquid state, which is used to cement the prosthetic component 1 to the device 10 and then to the patient's bone.

The invention thus conceived is susceptible of numerous modifications and variations, all falling within the scope of the inventive concept.

In addition, all details can be substituted by other technically equivalent elements. In practice, the materials used, as well as the contingent shapes and sizes, can be of any type in accordance with the requirements, without departing from the protective scope of the following claims.

What is claimed is:

1. A case-like device for a prosthetic component of a hip prosthesis or a shoulder prosthesis, the prosthetic component having a stem with a distal end and a connection end, and a perimetric flange extending outwards from the stem, the case-like device comprising:
a shaft having a part with a substantially tapered conformation towards a closing base, the part having a substantially conical or truncated cone or pyramid conformation and being sized to house the stem of the prosthetic component;
a flanged base extending outwards with respect to an opening of the device, the flanged base having an upper surface and a lower surface opposite the upper surface;
a cavity inside the device, the cavity having a space with a conformation corresponding to that of the shaft;
wherein the flanged base has a lateral extension substantially corresponding to or slightly greater than the perimetric flange of the stem, and the upper surface is adapted to abut and contact the perimetric flange.

2. The case-like device of claim 1, wherein the lower surface is adapted to abut and come into contact with a patient's bone, thereby avoiding contact between the bone and material of the prosthetic component.

3. The case-like device of claim 1, wherein the shaft has the same inclination and curvature as the stem of the prosthetic component.

4. The case-like device of claim 1, wherein the closing base may be inclined or not inclined with respect to a wall of the part of the shaft.

5. The case-like device of claim 1, wherein the device is made of a biocompatible plastic material selected from bone cement and PMMA.

6. The case-like device of claim 1, wherein the device has an internal metal core completely immersed in biocompatible material.

7. The case-like device of claim 1, wherein the device is completely made of biocompatible material without an internal metal core.

8. The case-like device of claim 1, wherein the device is impregnated with at least one active substance selected from an antibiotic and a medical agent.

9. The case-like device of claim 1, wherein the device is porous and equipped with through holes configured to allow bone cement to pass through.

10. The case-like device of claim 1, wherein the device is preformed and formed of a rigid material with predetermined sizes and thicknesses based on the type of prosthetic component to be housed.

11. A prosthetic system comprising:
a prosthetic component of a hip prosthesis or a shoulder prosthesis having a stem with a distal end and a connection end, a spherical or hemispherical or spherical cap head, a neck structure connecting the stem and the head, and a perimetric flange extending outwards from the stem; and
the case-like device according to claim 1.

12. The prosthetic system of claim 11, wherein the prosthetic component has housing seats and the case-like device has corresponding connection means configured to be housed in the housing seats.

13. The prosthetic system of claim 12, wherein the connection means have a substantially pin, cylinder, semi-cylinder, clip, C, ring or tubular shape.

14. The prosthetic system of claim 12, wherein the housing seats have a substantially circular cross section and the connection means have a substantially C-shaped cross section.

15. The prosthetic system of claim 11, further comprising bone cement in a fluid or liquid state for cementing the prosthetic component to the case-like device and to a patient's bone.

16. A method of implanting a hip or shoulder prosthesis comprising:
providing a prosthetic component having a stem with a perimetric flange;
providing the case-like device according to claim 1;
inserting the stem of the prosthetic component into the cavity of the case-like device such that the perimetric flange contacts the upper surface of the flanged base;
implanting the assembled prosthetic component and case-like device into a patient's bone such that the lower surface of the flanged base contacts the patient's bone.

17. The method of claim 16, further comprising applying bone cement between the case-like device and the patient's bone.

18. The method of claim 16, further comprising applying bone cement inside the cavity of the case-like device to bind the case-like device to the prosthetic component.

19. A kit comprising:
a prosthetic component having a stem and a head;
a case-like device comprising an opening and a cavity for receiving and housing at least said stem of said prosthetic component,
wherein said prosthetic component is made of a metal material, wherein said case-like device is placed between said prosthetic component and a bone of a patient to prevent the direct contact between said prosthetic component and the bone of the patient and to stably constrain said prosthetic component to the bone of the patient, wherein said device is made of a plastic material.

20. The kit according to claim 19, wherein said prosthetic component is a femoral component of a hip prosthesis or a humeral component of a shoulder prosthesis and wherein said head comprises a spherical or semi-spherical or spherical cap-shaped head, further comprising a neck-like structure having a cylindrical or truncated cone or pyramid shape adapted to connect said stem and said head, wherein said stem has a connection end connected to said neck-like structure, wherein said connection end departs from said neck-like structure in all directions and has a truncated cone or pyramid shape, up to said stem where a perimeter flange is present which extends towards the exterior of said prosthetic component and comprises a lower face.
